(19) United States
(12) Patent Application Publication
(10) Pub. No.: US 2023/0282354 A1
(43) Pub. Date: Sep. 7, 2023

(54) Cognitive Distortion Detection Method and System

(71) Applicant: Limbic Limited, London (GB)

(72) Inventors: Max Rollwage, London (GB); Ross Edward Francis Harper, London (GB)

(21) Appl. No.: 18/117,019

(22) Filed: Mar. 3, 2023

(30) Foreign Application Priority Data
Mar. 4, 2022 (GB) 2203075.3

(51) Int. Cl. G16H 50/20; G16H 80/00; G16H 20/70; G16H 10/20

(52) U.S. Cl. CPC G16H 50/20; G16H 10/20; G16H 20/70; G16H 80/00

(57) ABSTRACT

Systems and methods that provide for treatment of a cognitive disorder based on self-reporting of cognitive information.

### CROSS REFERENCE TO RELATED APPLICATION

This application is based upon and claims the benefit of and priority to prior United Kingdom Patent Application number 2203075.3 filed on 04 Mar. 2022, the entire contents of which is incorporated herein by reference.

### FIELD

The present invention relates to the automated screening and evaluation of self-reported medical therapy inputs from a user via a user interface and corresponding methods of treatment. More particularly, the present invention relates to the classification using trained computer models and the provision of substantially real-time feedback to the user inputting the self-reported therapy data via a user interface. Methods of treatment may make use of the feedback.

### BACKGROUND

Cognitive behavioural therapy focusses on the interaction between situations, thoughts, behaviours and emotions for our experiences. Following research over the previous decades, specific focus is put on the contribution of cognitive and behavioural patterns to the creation and persistence of mental health symptoms. The research has shown that a core pillar of cognitive behavioural therapy is the notion that it is not situations themselves that cause negative affect and ultimately poor well-being, but instead a person’s interpretation of these situations. As an example, if a friend does not invite us for dinner, it is not the fact that this friend has not invited us but the associated thought that the friend might not like us anymore that causes negative affect.

The main aim of cognitive behavioural therapy is to identify the influence of maladaptive cognition (i.e. thoughts) and behavioural patterns that cause a detriment to a patient’s mental health. In turn, treatment aims to restructure or correct these maladaptive patterns in order to improve the patient’s symptoms and well-being.

Thus, the way people process information, or the way they interpret situations, is a critical contributor to their mental health. Importantly, research has shown that people may not always interpret situations in an objective or accurate way and this faulty information processing is known as cognitive distortions (or thought distortions). For instance, a depressed patient might think in “extreme, negative, categorical, absolute, and judgmental” ways whereby these thinking errors contribute to the patient’s negative affect and thus to their mental health symptoms. Most critically, research has shown that these thinking errors lead to persistence of negative emotions even in the presence of contrary evidence and thus make mental health disorders more resistant to change. Hence, one of the core aims of cognitive behavioural therapy is to identify these thought distortions in order to challenge, change and restructure these mal-adaptive patterns.

The identification of mal-adaptive cognitive and behavioural patterns requires the monitoring of interactions between situations, thoughts, feelings, physiological responses and actions. In order to identify these interactions, it is a pre-requisite to recognise and record each of these factors in order to determine the interactions between them that contribute to the patient’s experience of any situation. For ease of reference, any exercises or techniques that require the patient/user to break down a situation into multiple components is referred to as a “self-monitoring” technique (although other terms are used in the literature).

However, by definition cognitive distortions are difficult to identify by the individual themselves. In this light, a critical first step is to teach the patient to identify the presence of these thought distortions in order to apply counteracting strategies when specific cognitive distortions occur. In order to identify potential thought distortions, self-monitoring techniques are used, where the patient monitors and records different components that contribute to the experience of a situation in, for instance, a three-column thought record is a common technique, where the patient differentiates between situation, emotion, thought. These techniques establish the habit to monitor own thoughts and the data collected through these exercises build the starting point for identifying potentially distorted thoughts.

During these self-monitoring techniques, the therapist helps and guides their patient in identifying the distinct factors contributing to the experience of a situation. The aim is to equip the patient with the competency to recognise these factors/components themselves and thus enable them with persisting skills to monitor and evaluate their own experiences. Thus, the therapist commonly acts in the role of a teacher or coach for a patient. The ability to conduct self-monitoring techniques in order to recognise and differentiate between situations, emotions, thoughts and behaviours is one of the fundamental skills practiced in cognitive behavioural therapy as the appropriate application of self-monitoring techniques enables more sophisticated intervention (e.g. the identification of cognitive distortions). Hence, the teaching of effective self-monitoring techniques builds the starting point for successful cognitive behavioural therapy and presents a pre-requisite for more advanced intervention techniques.

Therapists approach the provision of cognitive behavioural therapy with the following core facets:

1. Psychoeducation: explanation about the relation between situations, thoughts, emotions and behaviour in order to motivate the patient to engage in the monitoring of these components. Moreover, a critical part of psychoeducation is the explanation about how these components differ and giving clear examples for each of these components.

2. Self-monitoring techniques: Self-monitoring techniques provide a structure for the patient to record these different components. This structure can be as simple as providing a table with separate columns for situations, emotions, thoughts and behaviours. While there are many different techniques, the core unifying principle is a structure that prompts the patient to enter specific information in order to ensure that all components of interest have been reflected on and are recorded. These techniques encompass (but are not limited to) 3-column thought records (recording situation, feeling and thoughts), a 5-column thought record (recording situation, automatic thoughts, emotions, alternative thoughts and outcomes), worry logs (recording a situation, feeling and worry). Moreover, there are more specialized techniques that focus on the extraction of a “pure” versions of one of these components. For instance, the “downward arrow technique” asks repeated prompts in order to ensure that the person identifies a clear example of a thought. In summary, we refer to self-monitoring techniques for any technique that provides a structure which prompts the patient to record a specific type of information and supports the patient to clearly differentiate between different experiential components (i.e. situation, emotion, physiological response, thought, behaviour or outcome).

3. Evaluation: evaluating the adequacy of the recorded information and provide feedback: Most critically, the therapist has the role to review the information the patient has provided (e.g. the input to a 3-column thought record) and evaluate whether the patient has provided the desired form of information (e.g. inputted a clear thought where thoughts have been asked for). When a therapist detects that the patient has misidentified components, feedback will be given in order for the patient to learn the differentiation between these components. This feedback has two aims:
a. The feedback might refine the understanding of the specific situation discussed. Through this, self-monitoring helps the patient to clarify their experience of a specific situation (i.e. clearly identifying the situation, the resulting emotions and the associated thoughts that caused these emotions). This might be useful for further analysis of the experience, such as identifying distorted or maladaptive thought patterns.
b. Additionally, the feedback teaches the patient a generalizable skill of separating of these different components that influence the experience of a situation. Therefore, this feedback signal can be relevant for more general skill development and as such therapeutic in its own right.

Once the application of self-monitoring techniques is mastered by the patient, they can use these techniques for more sophisticated insights (e.g. a 3-column thought record might be used for identifying cognitive distortions).

However, identifying their own thought distortions is a challenging task for patients. Therefore, a therapist needs to first educate the patient about thought distortions (psychoeducation) and then assist the patient in identifying specific examples of thought distortions and giving feedback. The feedback about the presence of thought distortions has two reasons:

1. The patient realizes that a specific situation has been interpreted in a distorted and biased way might help them to reinterpret the situation. This provides an immediate remedy of the negative affect caused by the situation. Thus, the feedback might immediately improve the patient’s experienced emotions.

2. Providing feedback about the existence of thought distortions is a teaching signal that improves the patient’s own ability to identify thought distortions in the future and thus equips them with a generalizable skill.

Learning to detect own cognitive distortions is notoriously difficulty. Success in overcoming cognitive distortions depends on repeated feedback over extended periods of time. Providing this feedback is time intensive and feedback needs to be delivered by a highly trained individual (i.e. therapist). Moreover, this approach limits feedback to the therapy session, whereas immediate feedback appears to be critical for effectively counteracting this mental fluke according to research.

### SUMMARY OF INVENTION

Aspects and/or embodiments seek to provide a system that continuously evaluates patient thought records input via a user interface using machine learned models that evaluate and classify text input and provide feedback to the patient.

According to a first aspect, there is provided a computer-implemented method of providing a medical diagnosis by an automated system processing text input provided by a user via a user interface, comprising: receiving the text input via the user interface from the user, the text input comprising self-reported therapy data; processing the text input to determine one or more classifications using a trained computer model, the one or more classifications being selected from one or more predetermined classification groups, the one or more predetermined classification groups comprising a first group of classifications each predicting a specific medical diagnosis and a second group of classifications indicating a lack of a specific medical diagnosis, wherein the one or more classifications each indicate the medical diagnosis; and if the one or more classifications comprises any classifications from the first group of classifications, providing a notification to the user via the user interface wherein the notification is relevant to the one or more classifications from the first group of classifications, the notification comprising the medical diagnosis derived from the one or more classifications. Optionally, the specific medical diagnosis comprises one or more cognitive distortions. Optionally the first group of classifications each predicting a specific medical diagnosis comprises a first group of classifications each predicting a cognitive distortion and wherein the second group of classifications indicating a lack of a specific medical diagnosis comprises a second group of classifications indicating a lack of cognitive distortions.

Providing an automated system that enables the automatic screening and detection of distorted thinking, based on thought record input, can allow the generation of insights for the therapist as well as directly providing feedback to the patient. Through this, identifying cognitive distortions can be supported and trained in a substantially real-time, repeatable and scalable way.

Optionally, there is provided the step of requiring further input from the user via the user interface following display of the notification to the user via the user interface, wherein the further input comprises amendments to the text input.

Requiring input from the user to modify their original thought record can help train the user to improve their thinking processes.

Optionally, the first group of classifications comprise any or any combination of classifications predicting: catastrophizing; dichotomous thinking; negative filtering; fortune telling; mind reading; and/or personalising.

Various cognitive distortions can be classified by the trained models, including but not limited to: catastrophizing; dichotomous thinking; negative filtering; fortune telling; mind reading; and/or personalising; and other cognitive distortions that are described in the literature (and it should also be noted that other terminology may be used in the literature to refer to these example cognitive distortions).

Optionally, the notification to the user via the user interface comprises information relevant to the medical diagnosis derived from the one or more classifications.

Providing information relevant to the predicted diagnosis to the user entering the input data can allow the user access to pertinent information in a substantially real-time and impactful way that can improve their treatment effectiveness.

Optionally, the method further comprises preparing reporting data for a therapist and/or medical professional and outputting said reporting data.

Optionally, the text input comprises physiological data pertaining to the user. Optionally, the text input comprises thought record data.

The input data provided in the thought records can include relevant data about the user that can be used alongside thought data, such as the user noticing an increased heart rate or sweating when experiencing certain thoughts.

Optionally, there is also provided a database wherein the database comprises a plurality of medical diagnoses and related classification data and wherein the method further comprises retrieving one or more of said medical diagnoses using the determined one or more classifications and the related classification data in order to provide the retrieved one or more said medical diagnoses via the notification to the user.

The medical diagnoses may be stored in a database that can be accessed by the evaluation system to look up the relevant diagnosis for a classification made by the system, allowing the device to access a centrally maintained and updated source of data and also allowing the device to store further classification data in the database.

According to a further aspect, there is provided system operable to perform the method of any aspect, further comprising a user device operable to provide the user interface and receive the user text input and a server operable to receive the text input, process the text input and provide the notifications to the user device for display to the user.

### BRIEF DESCRIPTION OF DRAWINGS

Embodiments will now be described, by way of example only and with reference to the accompanying drawings having like-reference numerals, in which:

FIG. 1 shows an embodiment of a chatbot interface used to collect user input and prompt the user of a user device;

FIG. 2 shows an embodiment illustrating the process of collecting user input and monitoring the user input with an evaluation process; and

FIG. 3 shows an embodiment illustrating the process of evaluating user input and providing notifications dependent on the evaluation.

### SPECIFIC DESCRIPTION

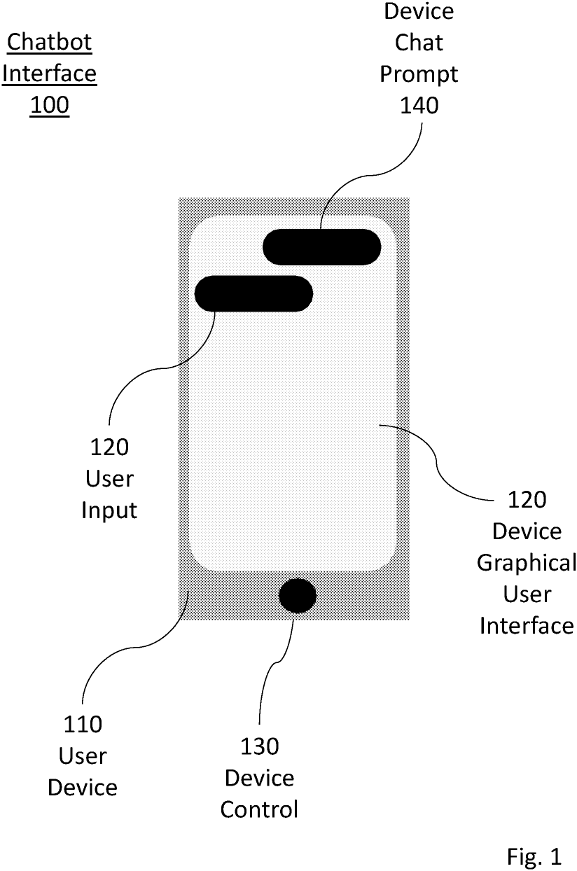

Referring to FIGS. 1 to 3, an embodiment of the text input and immediate diagnosis system will now be described below in further detail.

Referring first to FIG. 1, where a chatbot interface 100 is shown, in this embodiment, the solution is provided via a chatbot that presents prompts 140 to the user of a user device 110 and which allows responses 120 to be input by the user, typically in text format inputted via a physical device control 130 (or in some embodiments by voice) or more commonly as text input via a device graphical user interface 120 (for example, using an “on-screen” keyboard).

The user device 110 in this embodiment, as shown in FIG. 1, is a mobile smartphone or tablet computer, but other form factors and devices can be used in other embodiments including desktop computers, laptop computer, smart televisions or displays, augmented reality devices, virtual reality devices, smart speakers, etc.

The chatbot interface 100 can be used to both prompt the user to enter thought records at predetermined times, or at predetermined time intervals, or in response to certain activities performed on the user device 110 (e.g. after the completion of a voice or video call, or upon detecting certain keywords being entered via the user interface 120 or upon detection of certain physiological signals via sensors in the user device 110 or another device in communication with the user device 110 such as a smart watch or other physiological sensor).

In other embodiments, the exact delivery mechanism might not be limited to a chatbot environment but could easily be envisioned in any application which allows the patient to conduct thought record exercises in any computer-based setting.

This embodiment provides an automated system to support cognitive behavioural therapy patients, i.e. users, in the learning and performance of self-monitoring techniques. Self-monitoring describes the general approach of differentiating and recording different components that contribute to the experience of a situation (i.e. recording any of: a situation, emotion, thought, physiological response, behaviour) which is frequently used in cognitive behavioural therapy. The system of the embodiment uses natural language processing to automatically evaluate whether the patient has inputted the required form of information via the user interface (i.e. a pre-processing step to validate user input data) and optionally provides feedback to the user via a user interface if the data inputted is not in the correct form and/or requires the user to amend their input data until it meets the required form of information (i.e. the pre-processing step validates the data input as substantially of the correct input type or format). This allows the user/patient to refine the input to align the provided information with the required information and teaches the patient to accurately use self-monitoring techniques.

In this embodiment, the patient is initially presented with psychoeducational content via the device graphical user interface 120. In some embodiments the psychoeducational content is presented via the chatbot interface but in other embodiments it is presented as any of text documents, web pages, video content, interactive content or any other media that can be viewed on a user device 110.

This displayed psychoeducational content explains the concept of cognitive distortions and provides information and examples to the user about how these distorted thoughts contribute and sustain to symptoms of mental health disorders. Moreover, a critical component of psychoeducation is to present the patient with different forms of cognitive distortions (e.g. overgeneralization versus dichotomous thinking), and what the characteristics of these distortions are.

An additional psychoeducational component that can be displayed to users are specific strategies to counteract these distortions if they are identified (for instance, if a “shoulds and musts” distortion is identified, the patient might be suggested to apply more realistic and less perfectionistic expectations for their and other’s behaviours). However, in some embodiments, psychoeducational content about the counteracting strategies might not be delivered via a notification 140 until specific thought distortions have been identified.

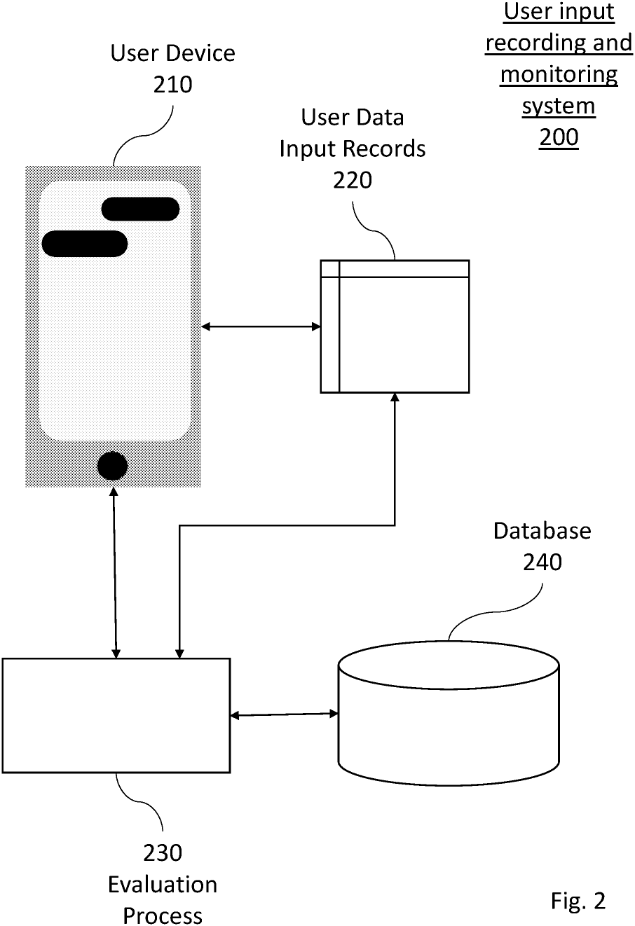

Referring to FIG. 2, a system for recording and monitoring user input 200 is presented and an embodiment will now be described in more detail below.

In this embodiment, a user device 210 (for example, a device 110 such as described in relation to FIG. 1 above) is used to display information and prompts to a user and receive data input by the user. Specifically, the data input by the user relates to and includes their thought records. The data input by the user, and other data collected by the device 210 and any other connected devices, is stored in a database 240 (in this embodiment, this database 240 is local to the user device 210 but it can be located remotely or distributed across multiple remote devices and optionally across the local device and one or more remote devices). An evaluation process 230 receives the user input either directly from the user device 210 or from the user data input records 220, as it can be in communication with either or both of the user device 210 and user data input records 220.

In order to identify distorted thoughts, data input by the user via device 210 needs to be systematically monitored and recorded. Here, in embodiments, different forms of thought records (e.g. 3- and 5-column thought records) and similar self-monitoring techniques (e.g. worry logs) are used in order to record the relevant information about the thoughts the patient is experiencing. It is critical that this acquired data represent represents clear examples of thoughts (i.e. not a mix of thoughts and other components) and is thus applicable to the examination of thought distortions. To ensure appropriate input data, the user input data might be screened with an additional machine-learning model which is trained to differentiate between different forms of self-reported information (e.g. report of situation, feelings, thoughts, behaviours and physiological experiences) to classify whether the user text input describes a thought. This additional screening might be applied as a pre-processing step to prompt the patient to input adequate information or discard examples that don’t represent thoughts for further analysis and this can then be stored in the user data input records 220. Any other collected data can be stored in the user data input records 220 as well, but this should be kept separate from the thought records in this embodiment. In other embodiments, all user data can be combined in the user data input records 220.

The evaluation process 230 performs the evaluation of the user input. In this embodiment, the evaluation process 230 comprises a trained deep learning model that is used to classify free-text input (i.e. the user data input capturing the user’s thoughts) as to whether the presented thought was distorted or not.

In some embodiments, a more complex version of the model is used in the evaluation process 230, where the algorithm predicts which (if any) specific cognitive distortion was present out of a list of the most common cognitive distortion (e.g. a commonly used list of cognitive distortions can include: catastrophizing; dichotomous thinking; negative filtering; fortune telling; mind reading; and personalising. Various cognitive distortions can be classified by the trained models, including but not limited to: catastrophizing; dichotomous thinking; negative filtering; fortune telling; mind reading; and/or personalising; and other cognitive distortions that are described in the literature (and it should also be noted that other terminology may be used in the literature to refer to these example cognitive distortions).

The model takes free-text as input, whereby this free-text is transformed into a vector representation. Many different ways of obtaining such vector representations are possible (e.g. term-frequency inverse document frequency or transformer networks) and the outlined application is independent of the used embedding of choice. This transformed text is then fed into the deep learning model in order to predict the probability that the user data that was input which represents the thought and which is being evaluated by the model contains one of the specified cognitive distortions.

In embodiments, the model output indicating the presence of a cognitive distortion might be used in one (or multiple) of the following ways:

1. Direct feedback to the user via the user interface of the user device 210 or by other notification means: if a distorted thought is predicted to be present in the user input data, this is immediately reported to the user and can be reported to the patient with additional information or with a requirement that the user provides further input. The reporting to the user can either be generic (i.e. independent of the type of distortion: e.g. “It looks like you are interpreting the situation in a very certain way, this might indicate a thought distortion. Do you think there might be other ways to see what happened?”) or specific for the distortion that occurred (e.g. presence of mind reading: “It looks like you are very certain about what other people think. This might indicate the thought distortion “mind reading”. Are you sure, you can be so certain about their views?”). This feedback component might include specific psychoeducation delivering tailored counteracting strategies for the experienced thought distortion. Such counteracting strategies have the aim to immediately help the person to reinterpret the experienced situation and through this reduce their distress. Moreover, this direct feedback will also act as teaching signal and help the patient learn to identify similar distortions in the future. Further, the user may optionally be required to provide further input, for example they may be prompted to amend their input to avoid the cognitive distortions predicted by the model.

2. Patients might repeatedly show the same cognitive distortion. Therefore, tracking the occurrence of distortions over time will be useful in order to give a user one or more insights about the forms of distortions that they most commonly experience and provide them with tailored psychoeducation specifically with information and counteracting strategies for the thought distortions they experience most commonly.

3. The specific thoughts containing distorted thinking might be flagged to the therapist in order to discuss the exact situation in a following therapy session.

4. The frequency of specific cognitive distortion might be presented to the therapist in order for them to keep track of this insight and discuss these observations in a following therapy session.

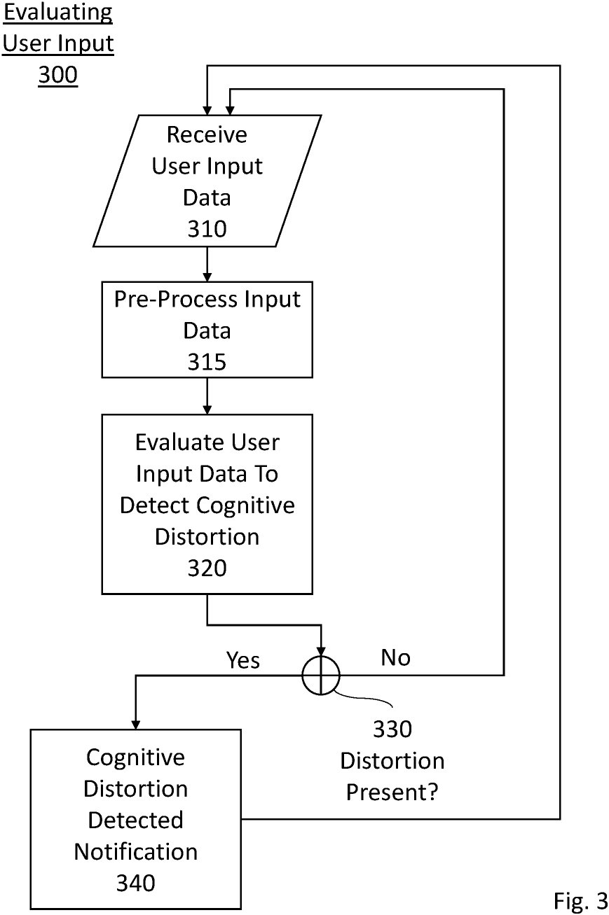

Referring next to FIG. 3, which shows an embodiment where the user input is evaluated 300, this embodiment will now be described in more detail below.

As in the previously described embodiments, user input data 310 is collected via a user interface from a user to populate a standard three-column thought record. After having been familiarized with this self-monitoring technique and having received some basic psychoeducational content regarding thought distortions, the patient is prompted to fill out the 3-column though record which is collected as the user input data 310. Specifically, the patient is presented with a prompt to describe (1) a situation that has recently caused them distress and (2) the emotion that has been caused by this situation: (1) “Can you tell me about a recent situation that has caused you distress?” (2) “How does that situation make you feel?”. Finally, the patient is asked for the associated thoughts that has contributed to the experienced emotion: “Now let’s see if we can identify the thought that is contributing to your feeling. What are you thinking about the situation that makes you feel this way?”

This embodiment only focuses on the detection of distorted versus non-distorted thoughts in the context of a 3-column thought record. There are multiple checks that are run as a pre-processing step 315 in order to ensure that the input data provided by the patient is suitable for the analysis to detect thought distortions, whereby the free-text input is transformed into a vector representation and fed into a deep neural network in order to classify whether the free-text input represents a thought or a different type of patient input (e.g. description of a situation or an emotion).

If the text input is deemed eligible for this analysis by the pre-processing step 315 (i.e. a clear thought, related to a distressing emotion), this text input is then evaluated by an evaluation process 320 comprising a deep learning algorithm which is trained to classify whether this thought present in the user input data 310 is distorted or not distorted. For this purpose, the text is transformed into two separate vector representations: one using a pre-trained sentence embedding (for example using the implementation described in the paper “sentence-BERT”, Reimers & Gurevych, 2019, see arXiv:1908.10084 which is incorporated by reference)); the other using a simple word-count embedding based on the most common keywords that are present in distorted thoughts.

The deep learning algorithm may be trained using training data. The training data may comprise historical patient records and may be used to pre-train weights of the deep learning algorithm. In some implementations, further data can be collected as the deep learning algorithm is used and further training can be performed, for example, by including patient records, clinical outcomes data and / or any digital biomarkers generated using any combination of natural language processing, analysis of typing patterns (as these data streams can be used to predict symptoms of medical conditions) and/or interactions with the system 200 (e.g. response times). Optionally instead or as well, other information can be used such as information collected passively by the user’s computing device including sensor data such as accelerometery, video, audio, temperature, light intensity, GPS, touch screen events, cursor movements, haptic feedback, electrocardiography, and / or photoplethysmography. In some embodiments, the training data comprises data associated with an individual or a group of individuals. A training data set may include data collected for a given individual (e.g., user data input records 220 comprising data for the individual collected using techniques described herein, such as self-reported therapy data provided by the user), and a deep learning algorithm (e.g., for the individual or other persons) may be trained using the individual training data set. This may enable generating deep learning algorithms that are tailored to trends for the individual. A training data set may include data collected for multiple individuals (e.g., user data input records 220 comprising data for the individuals collected using techniques described herein, such as self-reported therapy data provided by some or all of the users), and a deep learning algorithm (e.g., for some or all of the individuals in the group or other persons) may be trained using the group training data set. This may enable generating deep learning algorithms that benefit from a relatively large data set that incorporates general trends across multiple individuals.

The key word representation is derived by comparing the most common words for all classes of cognitive distortions compared to the most common words in non-distorted thoughts, whereby in this embodiment the 750 words are selected that appear most commonly in distorted thoughts but do not occur but do not occur in non-distorted thoughts (i.e. not in the top 2000 most common words). In other embodiments, different numbers of words can be used. The additional bag-of-words based embedding allows to capture qualitative difference for specific words used in distorted thinking. These two vector representations are fed into two separate streams of a neural network with separate hidden layers. These separate hidden layers are then combined into a combined final hidden layer which feeds into the output layer.

Applying this algorithm to the text input allows to evaluate whether the patient reported a distorted thought (or the probability of a thought distortion) and this is output by the evaluation process 320.

A check 330 is performed on the output of the evaluation process 320 to determine if a distortion is predicted to be present as the output from the evaluation process 320. If a distortion is predicted to be present, then a notification 340 is provided to the user else no notification is provided. In some embodiments, any relevant data records are updated accordingly (for example to store the user input data and a record indicating whether or not cognitive distortions are present).

The process of evaluating user input 300 may comprise collection of further user input data 310 and repetition of corresponding steps.

The process of evaluating user input 300 may comprise reporting results. The results may be reported to the user or a therapist (e.g., a therapist treating the user) or medical professional or other treatment personnel. For example, if a potentially distorted thought is detected, this thought may be flagged in a report for review by a therapist or medical professional in order to make them aware of the distortions their patients are experiencing. Moreover, the frequency (over time) of different distortions for each patient may be aggregated in the report in order to enable the therapist to track the occurrence of these distortions over time. In different embodiments, reports can be generated at regular frequencies or on-demand for the therapist or medical professional. The reports may comprise all data collected and any corresponding evaluations (e.g. the evaluations made at step 320). Alternatively, the reports may comprise a subset of the data collected and / or a subset of the evaluations. For example, prior to providing the reports, noise may be filtered out such that the reports contain key events only. In other words, reports may selectively provide diagnosis information to a therapist or medical professional.

An output of the process of evaluating user input 300 (i.e. a notification 340 or a report) may be provided as an input to action logic. The action logic be configured to select an action to perform responsive to the output and/or to cause performance of an action in response to the output. For example, as described above, the action may be transmission of the output to a user or a third party. The action logic may be configured to generate a treatment pathway (or “treatment plan”), schedule appointments with clinicians or to establish a communication with one or more third parties, such as a clinician or an emergency service or other treatment service. For example, the action logic may be configured to establish a communication channel between the user and a clinician or an emergency service or between a clinician and an emergency service and may transmit the output to the one or more third party. For example, a user may be allocated to a predetermined treatment pathway depending on any detected cognitive distortion. For example, allocation to a treatment pathway may be performed by the action logic. A predetermined treatment pathway is the route to which is patient is seen by a mental health care professional (e.g., by way of an in-person visit or virtual visit conducted by phone or video conference). There may be several different pre-programmed treatment pathways. For example, a treatment pathway for patients that are prioritised for early treatment so that they are seen by a mental health care professional within 2 weeks, or a treatment pathway for patients whose condition is relatively mild and who could be seen by a mental health care professional within a longer wait time of 8 weeks. The mental health care service may be informed of the user and their allocated treatment pathway (e.g., a schedule of the treatment plan) by the action logic. The user can then be seen by a mental health care professional according to their allocated treatment pathway. The action logic may be configured to prioritize some users for treatment based on the output.

As discussed above, the action logic may be configured to generate a treatment plan. Put differently, if a cognitive distortion is predicted to be present at step 330, a corresponding treatment plan may be generated. A treatment plan for an individual may include a listing of one or more parameters for treatment of the individuals. The parameters may include, for example, a schedule of treatment (e.g., a schedule of calls/visits to a mental health care professional), schedule of input (e.g., a schedule of prompting the individual for input of a specific type of information). Treatment of an individual may include treatment in accordance with parameters specified by a treatment plan for an individual (e.g., conducting calls/visits to a mental health care professional in accordance with a treatment plan schedule, prompting the individual for input of a specific type of information in accordance with the treatment plan schedule, and so forth). In some embodiments, a system (e.g., system 200) automatically schedules or contacts relevant parties for treatment in accordance with treatment plan parameters. For example, the system may automatically schedule an appointment for a meeting between an individual and a mental health care professional in accordance with a treatment plan schedule. A corresponding treatment plan may be generated upon a threshold number of distortions being predicted. The threshold may be with respect to all cognitive distortions or only specific cognitive distortions. In examples in which the threshold is with respect to specific cognitive distortions, the generated treatment plan may correspond to the specific cognitive distortions.

The process of evaluating user input may further comprise providing a portion of a treatment plan. For example, the user may be notified that the user should complete a session of the treatment plan. The user may be led through steps of the session. As an alternative example, the user may be notified that a call should be held between the user and a clinician.

As discussed above, the system 200 and / or the evaluation of user input 300 may be used for diagnosis and / or treatment of an individual user. Additionally or alternatively, the system 200 and / or the evaluation of user input 300 may be used to measure outcomes across multiple users. For example, the system 200 may monitor health outcomes of a plurality of users and the corresponding treatments each user has received. Accordingly, the system 200 may analyse this data and identify treatments which leads to positive health outcomes. The output of this analysis may be provided to clinicians and / or may be used for generating treatment plans. In some embodiments, the system includes one or more computing device configured to execute some or all of the operations described herein. In some embodiments, the system includes a non-transitory computer-readable storage medium comprising program instructions stored thereon that are executable by a processor (e.g., one or more processors of the system) to cause some or all of the operations described herein. Machine learning is the field of study where a computer or computers learn to perform classes of tasks using the feedback generated from the experience or data gathered that the machine learning process acquires during computer performance of those tasks.

Typically, machine learning can be broadly classed as using either supervised or unsupervised approaches, although there are particular approaches such as reinforcement learning and semi-supervised learning which have special rules, techniques and/or approaches.

Supervised machine learning is concerned with a computer learning one or more rules or functions to map between example inputs and desired outputs as predetermined by an operator or programmer, usually where a data set containing the inputs is labelled.

Unsupervised learning is concerned with determining a structure for input data, for example when performing pattern recognition, and typically uses unlabelled data sets.

Reinforcement learning is concerned with enabling a computer or computers to interact with a dynamic environment, for example when playing a game or driving a vehicle.

Various hybrids of these categories are possible, such as “semi-supervised” machine learning where a training data set has only been partially labelled. For unsupervised machine learning, there is a range of possible applications such as, for example, the application of computer vision techniques to image processing or video enhancement.

Unsupervised machine learning is typically applied to solve problems where an unknown data structure might be present in the data. As the data is unlabelled, the machine learning process is required to operate to identify implicit relationships between the data for example by deriving a clustering metric based on internally derived information. For example, an unsupervised learning technique can be used to reduce the dimensionality of a data set and attempt to identify and model relationships between clusters in the data set, and can for example generate measures of cluster membership or identify hubs or nodes in or between clusters (for example using a technique referred to as weighted correlation network analysis, which can be applied to high-dimensional data sets, or using k-means clustering to cluster data by a measure of the Euclidean distance between each datum).

Semi-supervised learning is typically applied to solve problems where there is a partially labelled data set, for example where only a subset of the data is labelled. Semi-supervised machine learning makes use of externally provided labels and objective functions as well as any implicit data relationships. When initially configuring a machine learning system, particularly when using a supervised machine learning approach, the machine learning algorithm can be provided with some training data or a set of training examples, in which each example is typically a pair of an input signal/vector and a desired output value, label (or classification) or signal. The machine learning algorithm analyses the training data and produces a generalised function that can be used with unseen data sets to produce desired output values or signals for the unseen input vectors/signals. The user needs to decide what type of data is to be used as the training data, and to prepare a representative real-world set of data. The user must however take care to ensure that the training data contains enough information to accurately predict desired output values without providing too many features (which can result in too many dimensions being considered by the machine learning process during training and could also mean that the machine learning process does not converge to good solutions for all or specific examples). The user must also determine the desired structure of the learned or generalised function, for example whether to use support vector machines or decision trees.

The use of unsupervised or semi-supervised machine learning approaches are sometimes used when labelled data is not readily available, or where the system generates new labelled data from unknown data given some initial seed labels.

Machine learning may be performed through the use of one or more of: a non-linear hierarchical algorithm; neural network; convolutional neural network; recurrent neural network; long short-term memory network; multi-dimensional convolutional network; a memory network; fully convolutional network or a gated recurrent network allows a flexible approach when generating the predicted block of visual data. The use of an algorithm with a memory unit such as a long short-term memory network (LSTM), a memory network or a gated recurrent network can keep the state of the predicted blocks from motion compensation processes performed on the same original input frame. The use of these networks can improve computational efficiency and also improve temporal consistency in the motion compensation process across a number of frames, as the algorithm maintains some sort of state or memory of the changes in motion. This can additionally result in a reduction of error rates.

Developing a machine learning system typically consists of two stages: (1) training and (2) production.

During the training the parameters of the machine learning model are iteratively changed to optimise a particular learning objective, known as the objective function or the loss.

Once the model is trained, it can be used in production, where the model takes in an input and produces an output using the trained parameters.

During the training stage of neural networks, verified inputs are provided, and hence it is possible to compare the neural network’s calculated output to then the correct the network is need be. An error term or loss function for each node in neural network can be established, and the weights adjusted, so that future outputs are closer to an expected result. Backpropagation techniques can also be used in the training schedule for the or each neural network.

The model can be trained using backpropagation and forward pass through the network. The loss function is an objective that can be minimised, it is a measurement between the target value and the model’s output.

The cross-entropy loss may be used. The cross-entropy loss is defined as

$${L}_{C E} = - {\sum }_{c = 1}^{C} y ∗ l o g \left(s\right)$$

where C is the number of classes, y ∈ {0,1} is the binary indicator for class c, and s is the score for class c.

In the multitask learning setting, the loss will consist of multiple parts. A loss term for each task,

$$L \left(x\right) = {λ}_{1} {L}_{1} + {λ}_{2} {L}_{2}$$

where L₁, L₂ are the loss terms for two different tasks and λ₁, λ₂, are weighting terms.

Any system feature as described herein may also be provided as a method feature, and vice versa. As used herein, means plus function features may be expressed alternatively in terms of their corresponding structure.

Any feature in one aspect may be applied to other aspects, in any appropriate combination. In particular, method aspects may be applied to system aspects, and vice versa. Furthermore, any, some and/or all features in one aspect can be applied to any, some and/or all features in any other aspect, in any appropriate combination.

It should also be appreciated that particular combinations of the various features described and defined in any aspects can be implemented and/or supplied and/or used independently.

Aspects of this disclosure may be better understood in view of the following enumerated embodiments:

1. A method for treatment of a cognitive disorder, the method comprising:
prompting, by a treatment system via a user interface of a user device, a user for a self-reporting of cognitive information for the user;
receiving, by a treatment system from the user via the user interface of the user device in response to the prompt, self-reported data indicative of a situation encountered by the user;
applying, by the treatment system, the self-reported therapy data to a trained computer model to determine a classification associated with the self-reported data;
determining, by the treatment system, that the classification corresponds to a diagnosis classification in a predetermined group of diagnosis classifications that are each associated with a predicted medical diagnosis;
in response to determining that the classification corresponds to the diagnosis classification in the predetermined group of classifications:
   determining, by the treatment system based on the diagnosis classification, a medical diagnosis for the user;
   providing, by the treatment system for presentation to the user via the user interface of a user device, a notification indicative of the diagnosis; and
   providing, by the treatment system to a therapist for use in treating a cognitive disorder of the user, a notification indicative of one or both of the diagnosis and the self-reported data indicative of the situation encountered by the user.

2. The method of embodiment 1, further comprising generating, by the treatment system based on the medical diagnosis, a treatment plan for the user, wherein the therapist treats the user for one or more cognitive disorders in accordance with the treatment plan.

3. A computer-implemented method of providing a medical diagnosis by an automated system processing text input provided by a user via a user interface, comprising:
receiving the text input via the user interface from the user, the text input comprising self-reported therapy data;
processing the text input to determine one or more classifications using a trained computer model, the one or more classifications being selected from one or more predetermined classification groups, the one or more predetermined classification groups comprising a first group of classifications each predicting a specific medical diagnosis and a second group of classifications indicating a lack of a specific medical diagnosis, wherein the one or more classifications each indicate the medical diagnosis; and
if the one or more classifications comprises any classifications from the first group of classifications, providing a notification to the user via the user interface wherein the notification is relevant to the one or more classifications from the first group of classifications, the notification comprising the medical diagnosis derived from the one or more classifications.

4. The method of embodiment 2, wherein the specific medical diagnosis comprises one or more cognitive distortions.

5. The method of embodiment 2, wherein the first group of classifications each predicting a specific medical diagnosis comprises a first group of classifications each predicting a cognitive distortion and wherein the second group of classifications indicating a lack of a specific medical diagnosis comprises a second group of classifications indicating a lack of cognitive distortions.

6. The method of embodiment 3, wherein the first group of classifications comprise any or any combination of classifications predicting: catastrophizing; dichotomous thinking; negative filtering; fortune telling; mind reading; and/or personalising.

7. The method of embodiment 2, further comprising the step of requiring further input from the user via the user interface following display of the notification to the user via the user interface, wherein the further input comprises amendments to the text input.

8. The method of embodiment 2, wherein the notification to the user via the user interface comprises information relevant to the medical diagnosis derived from the one or more classifications.

9. The method of embodiment 2, further comprising preparing reporting data for a therapist and/or medical professional and outputting said reporting data.

10. The method of embodiment 2, wherein the text input comprises physiological data pertaining to the user.

11. The method of embodiment 2, wherein the text input comprises thought record data.

12. The method of embodiment 2, further comprising a database wherein the database comprises a plurality of medical diagnoses and related classification data and wherein the method further comprises retrieving one or more of said medical diagnoses using the determined one or more classifications and the related classification data in order to provide the retrieved one or more said medical diagnoses via the notification to the user.

13. The method of embodiment 2, further comprising generating, by a treatment system based on the medical diagnosis, a treatment plan for the user, wherein the user is treated for the medical diagnosis in accordance with the treatment plan.

14. A system comprising one or more computing devices configured to:
receive, at an input of the one or more computing devices, a text input via a user interface from the user, the text input comprising self-reported therapy data;
process, at a processor of the one or more computing devices, the text input to determine one or more classifications using a trained computer model, the one or more classifications being selected from one or more predetermined classification groups, the one or more predetermined classification groups comprising a first group of classifications each predicting a specific medical diagnosis and a second group of classifications indicating a lack of a specific medical diagnosis, wherein the one or more classifications each indicate the medical diagnosis; and
if the one or more classifications comprises any classifications from the first group of classifications, providing, at an output of the one or more computing devices, a notification to the user via the user interface wherein the notification is relevant to the one or more classifications from the first group of classifications, the notification comprising the medical diagnosis derived from the one or more classifications.

15. A system comprising one or more computing devices configured to:
receive, at an input of the one or more computing devices, a text input via a user interface from the user, the text input comprising self-reported therapy data;
process, at a processor of the one or more computing devices, the text input to determine one or more classifications using a trained computer model, the one or more classifications being selected from one or more predetermined classification groups, the one or more predetermined classification groups comprising a first group of classifications each predicting a specific medical diagnosis and a second group of classifications indicating a lack of a specific medical diagnosis, wherein the one or more classifications each indicate the medical diagnosis; and
if the one or more classifications comprises any classifications from the first group of classifications, providing, at an output of the one or more computing devices, a notification to the user via the user interface wherein the notification is relevant to the one or more classifications from the first group of classifications, the notification comprising the medical diagnosis derived from the one or more classifications.

16. A method for treatment of a cognitive disorder, the method comprising:
prompting, by a treatment system via a user interface of a user device, a user for a self-reporting of cognitive information for the user;
receiving, by a treatment system from the user via the user interface of the user device in response to the prompt, self-reported data indicative of a situation encountered by the user;
applying, by the treatment system, the self-reported therapy data to a trained deep learning computer model to determine that the self-reported data is associated with a predicted cognitive distortion; and
in response to determining that the self-reported data is associated with a predicted cognitive distortion, providing, by the treatment system to a therapist for use in treating a cognitive disorder of the user, a notification indicative of one or both of the predicted cognitive distortion and the self-reported data indicative of the situation encountered by the user.

17. The method of embodiment 16, further comprising generating, by the treatment system based on the medical diagnosis, a treatment plan for the user, wherein the therapist treats the user for one or more cognitive disorders in accordance with the treatment plan.

1. A method for treatment of a cognitive disorder, the method comprising:
prompting, by a treatment system via a user interface of a user device, a user for a self-reporting of cognitive information for the user;
receiving, by a treatment system from the user via the user interface of the user device in response to the prompt, self-reported data indicative of a situation encountered by the user;
applying, by the treatment system, the self-reported therapy data to a trained computer model to determine a classification associated with the self-reported data;
determining, by the treatment system, that the classification corresponds to a diagnosis classification in a predetermined group of diagnosis classifications that are each associated with a predicted medical diagnosis;
in response to determining that the classification corresponds to the diagnosis classification in the predetermined group of classifications:
determining, by the treatment system based on the diagnosis classification, a medical diagnosis for the user;
providing, by the treatment system for presentation to the user via the user interface of a user device, a notification indicative of the diagnosis; and
providing, by the treatment system to a therapist for use in treating a cognitive disorder of the user, a notification indicative of one or both of the diagnosis and the self-reported data indicative of the situation encountered by the user.

2. A computer-implemented method of providing a medical diagnosis by an automated system processing text input provided by a user via a user interface, comprising:
receiving the text input via the user interface from the user, the text input comprising self-reported therapy data;
processing the text input to determine one or more classifications using a trained computer model, the one or more classifications being selected from one or more predetermined classification groups, the one or more predetermined classification groups comprising a first group of classifications each predicting a specific medical diagnosis and a second group of classifications indicating a lack of a specific medical diagnosis, wherein the one or more classifications each indicate the medical diagnosis; and
if the one or more classifications comprises any classifications from the first group of classifications, providing a notification to the user via the user interface wherein the notification is relevant to the one or more classifications from the first group of classifications, the notification comprising the medical diagnosis derived from the one or more classifications.

3. The method of claim 2, wherein the specific medical diagnosis comprises one or more cognitive distortions.

4. The method of claim 2, wherein the first group of classifications each predicting a specific medical diagnosis comprises a first group of classifications each predicting a cognitive distortion and wherein the second group of classifications indicating a lack of a specific medical diagnosis comprises a second group of classifications indicating a lack of cognitive distortions.

5. The method of either claim 3, wherein the first group of classifications comprise any or any combination of classifications predicting: catastrophizing; dichotomous thinking; negative filtering; fortune telling; mind reading; and/or personalising.

6. The method of claim 2, further comprising the step of requiring further input from the user via the user interface following display of the notification to the user via the user interface, wherein the further input comprises amendments to the text input.

7. The method of claim 2, wherein the notification to the user via the user interface comprises information relevant to the medical diagnosis derived from the one or more classifications.

8. The method of claim 2, further comprising preparing reporting data for a therapist and/or medical professional and outputting said reporting data.

9. The method of claim 2, wherein the text input comprises physiological data pertaining to the user.

10. The method of claim 2, wherein the text input comprises thought record data.

11. The method of claim 2, further comprising a database wherein the database comprises a plurality of medical diagnoses and related classification data and wherein the method further comprises retrieving one or more of said medical diagnoses using the determined one or more classifications and the related classification data in order to provide the retrieved one or more said medical diagnoses via the notification to the user.

12. A system comprising one or more computing devices configured to:
receive, at an input of the one or more computing devices, a text input via a user interface from the user, the text input comprising self-reported therapy data;
process, at a processor of the one or more computing devices, the text input to determine one or more classifications using a trained computer model, the one or more classifications being selected from one or more predetermined classification groups, the one or more predetermined classification groups comprising a first group of classifications each predicting a specific medical diagnosis and a second group of classifications indicating a lack of a specific medical diagnosis, wherein the one or more classifications each indicate the medical diagnosis; and
if the one or more classifications comprises any classifications from the first group of classifications, providing, at an output of the one or more computing devices, a notification to the user via the user interface wherein the notification is relevant to the one or more classifications from the first group of classifications, the notification comprising the medical diagnosis derived from the one or more classifications.

13. A system comprising one or more computing devices configured to:
receive, at an input of the one or more computing devices, a text input via a user interface from the user, the text input comprising self-reported therapy data;
process, at a processor of the one or more computing devices, the text input to determine one or more classifications using a trained computer model, the one or more classifications being selected from one or more predetermined classification groups, the one or more predetermined classification groups comprising a first group of classifications each predicting a specific medical diagnosis and a second group of classifications indicating a lack of a specific medical diagnosis, wherein the one or more classifications each indicate the medical diagnosis; and
if the one or more classifications comprises any classifications from the first group of classifications, providing, at an output of the one or more computing devices, a notification to the user via the user interface wherein the notification is relevant to the one or more classifications from the first group of classifications, the notification comprising the medical diagnosis derived from the one or more classifications.

14. A method for treatment of a cognitive disorder, the method comprising:
prompting, by a treatment system via a user interface of a user device, a user for a self-reporting of cognitive information for the user;
receiving, by a treatment system from the user via the user interface of the user device in response to the prompt, self-reported data indicative of a situation encountered by the user;
applying, by the treatment system, the self-reported therapy data to a trained deep learning computer model to determine that the self-reported data is associated with a predicted cognitive distortion; and
in response to determining that the self-reported data is associated with a predicted cognitive distortion, providing, by the treatment system to a therapist for use in treating a cognitive disorder of the user, a notification indicative of one or both of the predicted cognitive distortion and the self-reported data indicative of the situation encountered by the user.
